(19) United States
(12) Patent Application Publication
(10) Pub. No.: US 2023/0257814 A1
(43) Pub. Date: Aug. 17, 2023

(54) METHODS AND KITS FOR TREATING OR DIAGNOSING CANNABINOID HYPEREMESIS SYNDROME

(71) Applicant: CReDO Science, LLC, Austin, TX (US)

(72) Inventors: Ethan Russo, SW Vashon, WA (US); Venetia Whiteley, Austin, TX (US); Chris Spooner, Vernon (CA); Len May, Studio City, CA (US); Ryan Leslie, Yarrow Point, WA (US)

(21) Appl. No.: 17/905,021

(22) PCT Filed: Feb. 25, 2021
(86) PCT No.: PCT/US21/19724

§ 371 Date: Aug. 25, 2022

Related U.S. Application Data
(60) Provisional application No. 62/981,766, filed on Feb. 26, 2020.
(60) Provisional application No. 63/059,647, filed on Jul. 31, 2020.

(51) Int. Cl. C12Q 1/6883; C12Q 1/6858

(52) U.S. Cl. CPC C12Q 1/6883; C12Q 1/6858; C12Q 2600/156

(57) ABSTRACT

The invention relates to methods and kits for treating and/or diagnosing Cannabinoid hyperemesis syndrome (CHS) in a patient or for predicting propensity for or resistance to CHS in a cannabis user.

### CLAIM OF PRIORITY UNDER 35 U.S.C. § 119

The present Application for Patent claims priority to Provisional Application No. 62/981,766, entitled “METHODS AND KITS FOR TREATING OR DIAGNOSING CANNABINOID HYPEREMESIS SYNDROME” filed Feb. 26, 2020; and Provisional Application No. 63/059,647, entitled “METHODS AND KITS FOR TREATING OR DIAGNOSING CANNABINOID HYPEREMESIS SYNDROME” filed Jul. 31, 2020; both hereby expressly incorporated by reference herein,

### BACKGROUND

### Field

The invention relates to methods and kits for treating and/or diagnosing Cannabinoid hyperemesis syndrome (CHS) in a patient or for predicting propensity for or resistance to CHS in a cannabis user.

### Background

CHS is a constellation of symptoms and signs consisting of intractable vomiting, abdominal pain and hot bathing behavior. CHS solely occurs in the context of heavy chronic use of cannabis or extracts containing high amounts of tetrahydrocannabinol (THC). Diagnostic criteria have been recently tabulated (Sorensen, DeSanto, Borgelt, Phillips, & Monte, 2017): history of regular cannabis use for over 1 year (74.8%), severe nausea and vomiting (100%), vomiting that recurs in a cyclic pattern over months (100%), resolution of symptoms after stopping cannabis (96.8%), compulsive hot baths/showers with symptom relief (92.3%), male predominance (72.9%), abdominal pain (85.1%), at least weekly cannabis use (97.4%), and history of daily cannabis use (76.6%). The syndrome is increasingly identified, particularly in the USA. It is associated with frequent emergency visits for treatment and diagnosis, with high diagnostic expense ($30-90K) and general resistance to treatment with anti-emetics and analgesics. Considerable morbidity and even some fatalities have been reported.

The genomes of all organisms undergo spontaneous mutation in the course of their continuing evolution, generating variant forms of progenitor genetic sequences (Gusella, Ann. Rev. Biochem. 55, 831-854 (1986)). A variant form may confer an evolutionary advantage or disadvantage relative to a progenitor form or may be neutral. In some instances, a variant form confers an evolutionary advantage to the species and is eventually incorporated into the DNA of many or most members of the species and effectively becomes the most common form. Additionally, the effects of a variant form may be both beneficial and detrimental, depending on the circumstances. For example, a heterozygous sickle cell mutation confers resistance to malaria, but a homozygous sickle cell mutation is usually lethal. In many cases, both progenitor and variant forms survive and co-exist in a species population. The coexistence of multiple forms of a genetic sequence gives rise to genetic polymorphisms, including SNPs.

Approximately 90% of all polymorphisms in the human genome are SNPs. SNPs are single base positions in DNA at which different alleles, or alternative nucleotides, exist in a population.

The SNP position (interchangeably referred to herein as SNP, SNP site, SNP locus, SNP marker, or marker) is usually preceded by and followed by highly conserved sequences of the allele (e.g., sequences that vary in less than 1/100 or 1/1000 members of the populations). An individual may be homozygous or heterozygous for an allele at each SNP position. A SNP can, in some instances, be referred to as a “cSNP” to denote that the nucleotide sequence containing the SNP is an amino acid coding sequence. In some contexts, use herein of terms such as, “polymorphism”, “mutation”, “mutant”, “variation”, and “variant” can refer to a SNPs, as will be appreciated by persons of ordinary skill in the art.

A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion or deletion variant referred to as an “indel” (Weber et al., “Human diallelic insertion/deletion polymorphisms”, Am J Hum Genet 2002 October; 71(4):854-62).

A synonymous codon change, or silent mutation/SNP, is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. While SNPs can be bi-, tri-, or tetra-allelic, the vast majority of the SNPs are bi-allelic, and are thus often referred to as “bi-allelic markers”, or “di-allelic markers”.

As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases (Stephens et al. Science 293, 489-493, 20 Jul. 2001). As used herein, the term “haplotype” refers to a set of two or more alleles on a single chromosome. The term “diplotype” refers to a combination of two haplotypes that a diploid individual carries. The term “double diplotype”, also called “two-locus diplotype”, refers to a combination of diplotypes at two distinct loci for an individual.

### SUMMARY

The invention relates to the identification of a variant associated with CHS. The variant can be a variant sequence, marker or allele, such as a single nucleotide polymorphism (SNP). Some embodiments of the invention relate to the identification of a variant in the endocannabinoid system and/or neurotransmitter system. In some embodiments, the variant can be a mutation in the cytochrome P450 system, for example, with the CYP2C9 enzyme gene. In some embodiments, the invention relates to the identification of a variant associated with cannabinoid metabolism, such as the metabolism of THC. The variant(s) can be indicative of CHS-susceptibility or, in contrast, CHS-resistance.

The variant sequence, marker or allele can be of a gene selected from COMT, TRPV1, CYP2C9, CYP2C19, DRD2, CRY1 and/or ABCA1. In some embodiments, the variant can be associated with cannabinoid metabolism.

The marker can be a SNP haplotype or a SNP diplotype as set forth in Table 1. The SNP haplotype can be, for example, CGGC of COMT (rs4646316); CTTG of ABCA1 (rs2230806); ATGG of TRPV1 (rs879207); TCCC of DRD2 (rs4648318); CTTG of CYP2C9 (rs1934967); TCAA of TRPV1 (rs11655540); CCGG of COMT (rs165656); GCTT of CYP2C19 (rs4494250); and/or CTCG of CRY1 (rs2287161); or a marker in useful proximity thereto.

With reference to the RSIDs of the haplotypes recited above, the diplotypes can be in homozygous for some loci and heterozygous for others. Homozygous SNP diplotypes can be, for example, CTTG/CTTG of ABCA1; CTTG/CTTG of CYP2C9; and heterozygous diplotypes can be, for example, CGGC/TGGC of COMT; ATGG/GTGG of TRPV1; TCCC/CCCC of DRD2; TCAA/GCAA of TRPV1; CCGG/TCGG of COMT; GCTT/ACTT of CYP2C19; and/or GTCG/CTCG of CRY1; or a marker in useful proximity thereto.

The SNP allele can be an allele of a SNP selected from the group consisting of alleles reported under Reference SNP (rs) Report numbers: rs4646316, rs2230806, rs879207, rs4648318, rs1934967, rs11655540, rs165656, rs4494250, rs2287161, or a combination thereto.

Some embodiments of the invention relate to understanding the relationship of the TRPV1 receptor to the endocannabinoid system, the digestive system, or neurotransmitter function. For example, the invention relates to the association of a variant form of a TRPV1 receptor to propensity for or resistance to CHS in a patient, diagnosis of CHS in a patient, and/or type of treatment of CHS in a patient.

Receptor-level treatments for CHS are provided. The treatment can include, for example, competitive ligands of the variant receptors and can be in the form of inhaled, ingested, or topical treatments. For example, the patient can be treated with cutaneous application of TRPV1 agonists and/or desensitizers.

Methods of predicting propensity/resistance or diagnosing CHS in a patient with CHS symptoms are provided. The method can include obtaining a sample from the patient. The sample can be blood, saliva, or other body fluid. The method can include analyzing a sample for the presence of any single nucleotide polymorphisms (SNPs) or other markers that indicate a mutation possibly affecting TRPV1 receptors or endocannabinoid system and/or neurotransmitter system. The method can include comparing the SNPs or other mutations that affect TRPV1 receptors with genes in a healthy patient without CHS symptoms, wherein differences in sequence correlate with the presence of CHS.

The variants can be associated with a propensity of cannabis users to CHS, resistance of cannabis users to CHS, a positive diagnosis of CHS, a propensity of cannabis users to CHS.

In some embodiments, the combination of variants can be associated with a resistance of cannabis users to CHS, or a positive diagnosis of CHS.

In some embodiments, the gene associated with cannabinoid metabolism is at least one of COMT, TRPV1, CYP2C9, CYP2C19, DRD2, CRY1 and/or ABCA1.

Some embodiments of the invention relate to a method of treating patients suffering from CHS. The method can include diagnosing CHS as described herein. Treatment is determined based on findings of SNPs or other mutations of COMT, TRPV1, CYP2C9, CYP2C19, DRD2, and/or ABCA1, including but not necessarily limited to those listed in Table 1, or SNPS or other mutations that affect TRPV1 receptors or endocannabinoid system and/or neurotransmitter system.

In some embodiments, a patient with CHS and a receptor variant is treated with a ligand of the receptor capable of competitively displacing THC or otherwise interrupting the relationship between action of the variant receptor and the symptoms of CHS.

In some embodiments, a patient with CHS and a TRPV1 receptor variant is treated with a TRPV1 ligand capable of acting as an agonist/desensitizer. The ligand can be a natural compound such as compounds in ginger (Zingiber officinale).

Some embodiments of the invention relate to a kit for employing the methods disclosed herein. The kit can include regents for determining SNPs or other mutations of COMT, TRPV1, CYP2C9, CYP2C19, DRD2, CRY1 and/or ABCA1, including but not necessarily limited to those listed in Table 1, or SNPs or other mutations that affect TRPV1 receptors or cannabinoid metabolism or endocannabinoid system and/or neurotransmitter system in a patient. The same or a different kit can include effective treatments such as ligands for the variant receptor.

### BRIEF DESCRIPTION OF THE DRAWINGS

FIG. 1 depicts the location of rs4646316.

FIG. 2 depicts the location of rs1934967.

### DETAILED DESCRIPTION

Methods and kits for treating and/or diagnosing CHS in a patient or for predicting propensity for or resistance to CHS in a cannabis user are provided.

The present invention provides SNPs or other variants associated with CHS and related pathologies, nucleic acid molecules containing SNPs, methods and reagents for the detection of the SNPs disclosed herein, uses of these SNPs for the development of detection reagents, and assays or kits that utilize such reagents. The CHS-associated SNPs disclosed herein are useful for diagnosing, screening for, and evaluating predisposition to CHS, including an increased or decreased risk of developing CHS, the rate of progression of CHS, and related pathologies in humans. Furthermore, such SNPs and their encoded products can be useful targets for the development of therapeutic agents.

The present invention relates to the identification of novel SNPs, unique combinations of such SNPs, haplotypes or diplotypes of SNPs that are associated with CHS and in particular the increased or decreased risk of developing CHS. The polymorphisms disclosed herein are directly useful as targets for the design of diagnostic reagents and the development of therapeutic agents for use in the diagnosis and treatment of CHS and related pathologies.

Based on the identification of SNPs associated with CHS, the present invention also provides methods of detecting these variants as well as the design and preparation of detection reagents needed to accomplish this task. The invention specifically provides, for example, novel SNPs in genetic sequences involved in CHS and related pathologies, isolated nucleic acid molecules (including, for example, DNA and RNA molecules) containing these SNPs, variant proteins encoded by nucleic acid molecules containing such SNPs, antibodies to the encoded variant proteins, computer-based and data storage systems containing the novel SNP information, methods of detecting these SNPs in a test sample, methods of identifying individuals who have an altered (i.e., increased or decreased) risk of developing CHS based on the presence or absence of one or more particular nucleotides (alleles) at one or more SNP sites disclosed herein or the detection of one or more encoded variant products (e.g., variant mRNA transcripts or variant proteins), methods of identifying individuals who are more or less likely to respond to a treatment (or more or less likely to experience undesirable side effects from a treatment, etc.), methods of screening for compounds useful in the treatment of a disorder associated with a variant gene/protein, compounds identified by these methods, methods of treating disorders mediated by a variant gene/protein, methods of using the novel SNPs of the present invention for human identification, etc.

The present invention provides novel SNPs associated with CHS and related pathologies, as well as SNPs that were previously known in the art, but were not previously known to be associated with CHS. Accordingly, the present invention provides novel compositions and methods based on the novel SNPs disclosed herein, and also provides novel methods of using the known, but previously unassociated, SNPs in methods relating to CHS (e.g., for diagnosing CHS, etc.).

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers may be designed to hybridize to either strand and SNP genotyping methods disclosed herein may generally target either strand. Throughout the specification, in identifying a SNP position, reference is generally made to the protein-encoding strand, only for the purpose of convenience.

References to variant peptides, polypeptides, or proteins of the present invention include peptides, polypeptides, proteins, or fragments thereof, that contain at least one amino acid residue that differs from the corresponding amino acid sequence of the art-known peptide/polypeptide/protein (the art-known protein may be interchangeably referred to as the “wild-type”, “reference”, or “normal” protein). Such variant peptides/polypeptides/proteins can result from a codon change caused by a nonsynonymous nucleotide substitution at a protein-coding SNP position (i.e., a missense mutation) disclosed by the present invention. Variant peptides/polypeptides/proteins of the present invention can also result from a nonsense mutation, i.e., a SNP that creates a premature stop codon, a SNP that generates a read-through mutation by abolishing a stop codon, or due to any SNP disclosed by the present invention that otherwise alters the structure, function/activity, or expression of a protein, such as a SNP in a regulatory region (e.g. a promoter or enhancer) or a SNP that leads to alternative or defective splicing, such as a SNP in an intron or a SNP at an exon/intron boundary. As used herein, the terms “polypeptide”, “peptide”, and “protein” may be used interchangeably unless specific context would indicate otherwise.

In a specific embodiment of the present invention, SNPs that occur naturally in the human genome are provided as isolated nucleic acid molecules. These SNPs are associated with CHS and related pathologies. In particular the SNPs are associated with either an increased or decreased risk of developing CHS. As such, they can have a variety of uses in the diagnosis and/or treatment of CHS and related pathologies. In some embodiments, a nucleic acid of the invention is an amplified polynucleotide, which is produced by amplification of a SNP-containing nucleic acid template. In another embodiment, the invention provides for a variant protein that is encoded by a nucleic acid molecule containing a SNP disclosed herein.

In another embodiment of the invention, a reagent for detecting a SNP in the context of its naturally-occurring flanking nucleotide sequences (which can be, e.g., either DNA or mRNA) is provided. In particular, such a reagent may be in the form of, for example, a hybridization probe or an amplification primer that is useful in the specific detection of a SNP of interest. In an alternative embodiment, a protein detection reagent is used to detect a variant protein that is encoded by a nucleic acid molecule containing a SNP disclosed herein. In another embodiment, a protein detection reagent is an antibody or an antigen-reactive antibody fragment.

Various embodiments of the invention also provide kits comprising SNP detection reagents, and methods for detecting the SNPs disclosed herein by employing detection reagents. In some embodiments, the present invention provides for a method of identifying an individual having an increased or decreased risk of developing CHS by detecting the presence or absence of one or more SNP alleles disclosed herein.

In another embodiment, a method for diagnosis of CHS and related pathologies by detecting the presence or absence of one or more SNP alleles disclosed herein is provided. In another embodiment, the invention provides for a method of identifying an individual having an altered (either increased, or, decreased) risk of developing CHS by detecting the presence or absence of one or more SNP haplotypes, diplotypes, or multi-locus diplotypes (diplotypes of two or more loci) disclosed herein.

As aspect of the invention is a method for diagnosing Cannabinoid hyperemesis syndrome (CHS) in a patient that can include obtaining a sample from a patient; analyzing the sample for a variant sequence, marker or allele of one or more genes, wherein the presence of the variant sequence, marker or allele indicates a likelihood of CHS; and diagnosing CHS based upon presence of the variant sequence, marker, or allele.

Table 1 provides genomic information of SNPs that can be used in the present invention.

TABLE 1

| Gene | RSID | Mutation | Allele | Zygosity | Diplotype | Haplotype |
|---|---|---|---|---|---|---|
| COMT | rs4646316 | Intron | C > T | Heterozygous | CGGC/TGGC | CGGC |
| ABCA1 | rs2230806 | Synonymous | C > T | Homozygous | CTTG/CTTG | CTTG |
| TRPV1 | rs879207 | Downstream | A > G | Heterozygous | ATGG/GTGG | ATGG |
| DRD2 | rs4648318 | Intron | T > C | Heterozygous | TCCC/CCCC | TCCC |
| CYP2C9 | rs1934967 | Intron | C > T | homozygous | CTTG/CTTG | CTTG |
| TRPV1 | rs11655540 | Intron | T > G | Heterozygous | TCAA/GCAA | TCAA |
| COMT | rs165656 | Intron | C > T | Heterozygous | CCGG/TCGG | CCGG |
| CYP2C19 | rs4494250 | Intron | G > A | Heterozygous | GCTT/ACTT | GCTT |
| CRY1 | rs2287161 | downstream | G > C | Heterozygous | GTCG/CTCG | GTCG |

As shown in Table 1, the variant can be, for example, in a gene selected from COMT, TRPV1, CYP2C9, CYP2C19, DRD2, CRY1 and/or ABCA1. In other embodiments, the variant can be associated with the endocannabinoid system and/or neurotransmitter system.

The SNP haplotype can be, for example, CGGC of COMT; ATGG or TRPV1; CTTG of CYP2C9; TCCC of DRD2; and/or CTTG of ABCA1, including but not necessarily limited to those listed in Table 1, or a marker in useful proximity thereto.

The SNP diplotype can be, for example, CGGC/TGGC of COMT; ATGG/GTGG of TRPV1; CTTG/CTTG of CYP2C9; TCCC/CCCC of DRD2; and/or CTTG/CTTG of ABCA1, including but not necessarily limited to those listed in Table 1, or a marker in useful proximity thereto.

The SNP allele can be an allele of a SNP selected from the group consisting of rs4646316, rs879207, rs1934967, rs4648318, rs2230806, rs11655540, rs165656, rs113934938, or a combination of any number of them.

The nucleic acid molecules of the invention can be inserted in an expression vector, such as to produce a variant protein in a host cell. Thus, the present invention also provides for a vector comprising a SNP-containing nucleic acid molecule, genetically-engineered host cells containing the vector, and methods for expressing a recombinant variant protein using such host cells. In another specific embodiment, the host cells, SNP-containing nucleic acid molecules, and/or variant proteins can be used as targets in a method for screening and identifying therapeutic agents or pharmaceutical compounds useful in the treatment of CHS and related pathologies.

An aspect of this invention is a method for treating CHS in a human subject wherein said human subject harbors a variant in COMT, TRPV1, CYP2C9, CYP2C19, DRD2, CRY1 and/or ABCA1, which method comprises administering to said human subject a therapeutically or prophylactically effective amount of one or more agents counteracting the effects of the disease, such as by inhibiting (or stimulating) the activity of COMT, TRPV1, CYP2C9, CYP2C19, DRD2, CRY1 and/or ABCA1. Treatments include a dosing protocol sufficient to counter the effects of the disease.

Receptor-level treatments for CHS are also provided. The treatment can include, for example, competitive ligands of the variant receptors and can be in the form of inhaled, ingested, or topical treatments. For example, the patient can be treated with cutaneous application of TRPV1 agonists and/or desensitizers.

Another aspect of this invention is a method for identifying an agent useful in therapeutically or prophylactically treating CHS and related pathologies in a human subject wherein said human subject harbors variant of a gene identified in Table 1, which method comprises contacting the gene, transcript, or encoded protein with a candidate agent under conditions suitable to allow formation of a binding complex between the gene, transcript, or encoded protein and the candidate agent and detecting the formation of the binding complex, wherein the presence of the complex identifies said agent.

Another aspect of the invention is a method for developing a personalized treatment protocol for a patient with CHS including determining the genotype of the patient and recommending treatment based on the genotype, wherein the treatment can be selected from stopping cannabis consumption, administration of medication, and/or a traditional remedy.

Another aspect of this invention is a method for treating CHS and related pathologies in a human subject including determining that said human subject harbors a variant in COMT, TRPV1, CYP2C9, CYP2C19, DRD2, and/or ABCA1, and administering to said subject a therapeutically or prophylactically effective amount of one or more agents counteracting the effects of the disease.

Yet another aspect of this invention is a method for evaluating the suitability of a patient for CHS treatment comprising determining the genotype of said patient with respect to a particular set of SNP markers, said SNP markers comprising a plurality of individual SNPs ranging from 1 or more variants in COMT, TRPV1, CYP2C9, CYP2C19, DRD2, CRY1 and/or ABCA1, and calculating a score using an appropriate algorithm based on the genotype of said patient, the resulting score being indicative of the suitability of said patient undergoing CHS treatment.

Another aspect of the invention is a method of treating a CHS patient comprising administering an appropriate drug in a therapeutically effective amount to said CHS patient whose genotype has been shown to contain a plurality of SNPs selected from a variant in COMT, TRPV1, CYP2C9, CYP2C19, DRD2, CRY1 and/or ABCA1.

### Kits

Some embodiments of the invention relate to a kit for employing the methods disclosed herein. The kit can include reagents for determining SNPs or other mutations of COMT, TRPV1, CYP2C9, CYP2C19, DRD2, CRY1 and/or ABCA1, including but not necessarily limited to those listed in Table 1, or SNPs or other mutations that affect TRPV1 receptors or cannabinoid metabolism in a patient. Reaction reagents can include a detection reagent that specifically detects a specific target SNP position disclosed herein, and that is preferably specific for a particular nucleotide (allele) of the target SNP position (i.e., the detection reagent preferably can differentiate between different alternative nucleotides at a target SNP position, thereby allowing the identity of the nucleotide present at the target SNP position to be determined). Typically, such detection reagent hybridizes to a target SNP-containing nucleic acid molecule by complementary base-pairing in a sequence specific manner, and discriminates the target variant sequence from other nucleic acid sequences such as an art-known form in a test sample. An example of a detection reagent is a probe that hybridizes to a target nucleic acid containing one or more of the SNPs provided herein. In a preferred embodiment, such a probe can differentiate between nucleic acids having a particular nucleotide (allele) at a target SNP position from other nucleic acids that have a different nucleotide at the same target SNP position. Another example of a detection reagent is a primer which acts as an initiation point of nucleotide extension along a complementary strand of a target polynucleotide. The SNP sequence information provided herein is also useful for designing primers, e.g. allele-specific primers, to amplify (e.g., using PCR) any SNP of the present invention.

A person skilled in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art. The terms “kits” and “systems”, as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). Accordingly, the present invention further provides SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more SNPs of the present invention. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment of the present invention, kits are provided which contain the necessary reagents to carry out one or more assays to detect one or more SNPs disclosed herein. In a preferred embodiment of the present invention, SNP detection kits/systems are in the form of nucleic acid arrays, or compartmentalized kits, including microfluidic/lab-on-a-chip systems.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is a SNP of the present invention. In some kits/systems, the allele-specific probes are immobilized to a substrate such as an array or bead. For example, the same substrate can comprise allele-specific probes for detecting at least 1, 2, 3, 4, 5, 6, 7, 8 or all of the SNPs disclosed herein.

The terms “arrays”, “microarrays”, and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522, which is herein incorporated by reference in its entirety for all purposes.

Nucleic acid arrays are reviewed in the following references: Zammatteo et al., “New chips for molecular biology and diagnostics”, Biotechnol Annu Rev. 2002; 8:85-101; Sosnowski et al., “Active microelectronic array system for DNA hybridization, genotyping and pharmacogenomic applications”, Psychiatr Genet. 2002 December; 12(4): 181-92; Heller, “DNA microarray technology: devices, systems, and applications”, Annu Rev Biomed Eng. 2002; 4:129-53. Epub 2002 Mar. 22; Kolchinsky et al., “Analysis of SNPs and other genomic variations using gel-based chips”, Hum Mutat. 2002 April; 19(4):343-60; and McGall et al., “High-density genechip oligonucleotide probe arrays”, Adv Biochem Eng Biotechnol. 2002; 77:21-42, each of which is herein incorporated by reference in its entirety for all purposes.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length. The microarray or detection kit can contain polynucleotides that cover the known 5′ or 3′ sequence of a gene/transcript or target SNP site, sequential polynucleotides that cover the full-length sequence of a gene/transcript; or unique polynucleotides selected from particular areas along the length of a target gene/transcript sequence, particularly areas corresponding to one or more SNPs disclosed herein. Polynucleotides used in the microarray or detection kit can be specific to a SNP or SNPs of interest (e.g., specific to a particular SNP allele at a target SNP site, or specific to particular SNP alleles at multiple different SNP sites), or specific to a polymorphic gene/transcript or genes/transcripts of interest.

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions may be preferable when using, for example, nucleic acid arrays of allele-specific probes for SNP detection. Such high stringency conditions are described in the preceding section, and are well known to those skilled in the art and can be found in, for example, Current Protocols in Molecular Biology, John Wiley & Sons, New York (1989), 6.3.1-6.3.6, which is herein incorporated by reference in its entirety for all purposes.

In other embodiments, the arrays are used in conjunction with chemiluminescent detection technology. The following patents and patent applications, which are all hereby incorporated by reference, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5,994,073, 5,981,768, 5,871,938, 5,843,681, 5,800,999, and 5,773,628 describe methods and compositions of dioxetane for performing chemiluminescent detection; and U.S. published application US2002/0110828 discloses methods and compositions for microarray controls.

Using such arrays or other kits/systems, the present invention provides methods of identifying the SNPs disclosed herein in a test sample. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

A SNP detection kit/system of the present invention may include components that are used to prepare nucleic acids from a test sample for the subsequent amplification and/or detection of a SNP-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens. The test samples used in the above-described methods will vary based on such factors as the assay format, nature of the detection method, and the specific tissues, cells or extracts used as the test sample to be assayed. Methods of preparing nucleic acids, proteins, and cell extracts are well known in the art and can be readily adapted to obtain a sample that is compatible with the system utilized. Automated sample preparation systems for extracting nucleic acids from a test sample are commercially available, and examples are Qiagen's BioRobot 9600, Applied Biosystems' PRISM™ 6700 sample preparation system, and Roche Molecular Systems' COBAS AmpliPrep System.

Another form of kit contemplated by the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting one or more SNPs of the present invention, one or more containers which contain wash reagents (such as phosphate buffered saline, Tris-buffers, etc.), and one or more containers which contain the reagents used to reveal the presence of the bound probe or other SNP detection reagents. The kit can optionally further comprise compartments and/or reagents for, for example, nucleic acid amplification or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art (see, e.g., Weigl et al., “Lab-on-a-chip for drug development”, Adv Drug Deliv Rev. 2003 Feb. 24; 55(3):349-77), which is herein incorporated by reference in its entirety for all purposes. In such microfluidic devices, the containers may be referred to as, for example, microfluidic “compartments”, “chambers”, or “channels”.

Microfluidic devices, which may also be referred to as “lab-on-a-chip” systems, biomedical micro-electro-mechanical systems (bioMEMs), or multicomponent integrated systems, are exemplary kits/systems of the present invention for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more SNPs of the present invention. One example of a microfluidic system is disclosed in U.S. Pat. No. 5,589,136, which is herein incorporated by reference in its entirety for all purposes, and which describes the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. Nos. 6,153,073, Dubrow et al., and 6,156,181, Parce et al, each of which is herein incorporated by reference in its entirety for all purposes.

For genotyping SNPs, an exemplary microfluidic system may integrate, for example, nucleic acid amplification, primer extension, capillary electrophoresis, and a detection method such as laser induced fluorescence detection. In a first step of an exemplary process for using such an exemplary system, nucleic acid samples are amplified, preferably by PCR. Then, the amplification products are subjected to automated primer extension reactions using ddNTPs (specific fluorescence for each ddNTP) and the appropriate oligonucleotide primers to carry out primer extension reactions which hybridize just upstream of the targeted SNP. Once the extension at the 3′ end is completed, the primers are separated from the unincorporated fluorescent ddNTPs by capillary electrophoresis. The separation medium used in capillary electrophoresis can be, for example, polyacrylamide, polyethyleneglycol or dextran. The incorporated ddNTPs in the single nucleotide primer extension products are identified by laser-induced fluorescence detection. Such an exemplary microchip can be used to process, for example, at least 96 to 384 samples, or more, in parallel.

The same or a different kit can include effective treatments. For example, the kit can include ligands for a variant receptor.

### SNP Genotyping Methods

The process of determining which specific nucleotide (i.e., allele) is present at each of one or more SNP positions, such as a SNP position in a nucleic acid molecule disclosed herein, is referred to as SNP genotyping. The present invention provides methods of SNP genotyping, such as for use in screening for CHS or related pathologies, or determining predisposition thereto, or determining responsiveness to a form of treatment, or in genome mapping or SNP association analysis, etc.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues Mol Biol. 2003 April; 5(2):43-60; Shi, “Technologies for individual genotyping: detection of genetic polymorphisms in drug targets and disease genes”, Am J Pharmacogenomics. 2002; 2(3):197-205; and Kwok, “Methods for genotyping single nucleotide polymorphisms”, Annu Rev Genomics Hum Genet 2001; 2:235-58. Exemplary techniques for high-throughput SNP genotyping are described in Marnellos, “High-throughput SNP analysis for genetic association studies”, Curr Opin Drug Discov Devel. 2003 May; 6(3):317-21, each of which is herein incorporated by reference in its entirety for all purposes. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Each of the foregoing references is herein incorporated by reference in its entirety for all purposes. Sequence variations at specific locations can also be assessed by nuclease protection assays such as RNase and S1 protection or chemical cleavage methods.

In a preferred embodiment, SNP genotyping is performed using the TaqMan assay, which is also known as the 5′ nuclease assay (U.S. Pat. Nos. 5,210,015 and 5,538,848, which are herein incorporated by reference in their entireties for all purposes). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in diagnostic assays for CHS and related pathologies, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

Another preferred method for genotyping the SNPs of the present invention is the use of two oligonucleotide probes in an OLA (see, e.g., U.S. Pat. No. 4,988,617, which is fully incorporated by reference herein). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of OLA: U.S. Pat. Nos. 6,027,889, 6,268,148, 5494810, 5830711, and 6054564 describe OLA strategies for performing SNP detection; WO 97/31256 and WO 00/56927 describe OLA strategies for performing SNP detection using universal arrays, wherein a zipcode sequence can be introduced into one of the hybridization probes, and the resulting product, or amplified product, hybridized to a universal zip code array; U.S. application US01/17329 (and Ser. No. 09/584,905) describes OLA (or LDR) followed by PCR, wherein zipcodes are incorporated into OLA probes, and amplified PCR products are determined by electrophoretic or universal zipcode array readout; U.S. applications 60/427,818, 60/445,636, and 60/445,494 describe SNPlex methods and software for multiplexed SNP detection using OLA followed by PCR, wherein zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

Typically, the primer extension assay involves designing and annealing a primer to a template PCR amplicon upstream (5′) from a target SNP position. A mix of dideoxynucleotide triphosphates (ddNTPs) and/or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer.

The extended primers can then be purified and analyzed by MALDI-TOF mass spectrometry to determine the identity of the nucleotide present at the target SNP position. In one method of analysis, the products from the primer extension reaction are combined with light absorbing crystals that form a matrix. The matrix is then hit with an energy source such as a laser to ionize and desorb the nucleic acid molecules into the gas-phase. The ionized molecules are then ejected into a flight tube and accelerated down the tube towards a detector. The time between the ionization event, such as a laser pulse, and collision of the molecule with the detector is the time of flight of that molecule. The time of flight is precisely correlated with the mass-to-charge ratio (m/z) of the ionized molecule. Ions with smaller m/z travel down the tube faster than ions with larger m/z and therefore the lighter ions reach the detector before the heavier ions. The time-of-flight is then converted into a corresponding, and highly precise, m/z. In this manner, SNPs can be identified based on the slight differences in mass, and the corresponding time of flight differences, inherent in nucleic acid molecules having different nucleotides at a single base position. For further information regarding the use of primer extension assays in conjunction with MALDI-TOF mass spectrometry for SNP genotyping, see, e.g., Wise et al., “A standard protocol for single nucleotide primer extension in the human genome using matrix-assisted laser desorption/ionization time-of-flight mass spectrometry”, Rapid Commun Mass Spectrom. 2003; 17(11):1195-202.

The following references provide further information describing mass spectrometry-based methods for SNP genotyping: Bocker, “SNP and mutation discovery using base-specific cleavage and MALDI-TOF mass spectrometry”, Bioinformatics. 2003 July; 19 Suppl 1:144-153; Storm et al., “MALDI-TOF mass spectrometry-based SNP genotyping”, Methods Mol Biol. 2003; 212:241-62; Jurinke et al., “The use of Mass ARRAY technology for high throughput genotyping”, Adv Biochem Eng Biotechnol. 2002; 77:57-74; and Jurinke et al., “Automated genotyping using the DNA MassArray technology”, Methods Mol Biol. 2002; 187:179-92, each of which is herein incorporated by reference in its entirety for all purposes.

SNPs can also be scored by direct DNA sequencing. A variety of automated sequencing procedures can be utilized ((1995) Biotechniques 19:448), including sequencing by mass spectrometry (see, e.g., PCT International Publication No. WO94/16101; Cohen et al., Adv. Chromatogr. 36:127-162 (1996); and Griffin et al., Appl. Biochem. Biotechnol. 38:147-159 (1993), each of which is herein incorporated by reference in its entirety for all purposes.). The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730×1 DNA Analyzers (Foster City, Calif.), is commonly used in the art for automated sequencing.

Other methods that can be used to genotype the SNPs of the present invention include single-strand conformational polymorphism (SSCP), and denaturing gradient gel electrophoresis (DGGE) (Myers et al., Nature 313:495 (1985), which is fully incorporated by reference herein). SSCP identifies base differences by alteration in electrophoretic migration of single stranded PCR products, as described in Orita et al., Proc. Nat. Acad. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel (Erlich, ed., PCR Technology, Principles and Applications for DNA Amplification, W. H. Freeman and Co, New York, 1992, Chapter 7).

Sequence-specific ribozymes (U.S. Pat. No. 5,498,531, which is fully incorporated by reference herein) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

SNP genotyping is useful for numerous practical applications, as described below. Examples of such applications include, but are not limited to, SNP-disease association analysis, disease predisposition screening, disease diagnosis, disease prognosis, disease progression monitoring, determining therapeutic strategies based on an individual's genotype (“pharmacogenomics”), developing therapeutic agents based on SNP genotypes associated with a disease or likelihood of responding to a drug, stratifying a patient population for clinical trial for a treatment regimen, predicting the likelihood that an individual will experience toxic side effects from a therapeutic agent, and human identification applications such as forensics.

### COMT

COMT, or catechol-o-methyltranferase, is the enzyme that catabolizes catecholamines, epinephrine, norepinephrine, and especially, dopamine. Conditions of dopamine excess, as encountered in pharmacotherapy with dopamine agonist such as L-dopa and bromocriptine are associated with induction of compulsive behavior including gambling, sex addiction and substance abuse, particularly alcoholism.

Mutations of the gene encoding COMT have been investigated, and specifically including this RSID (reference snp [single-nucleotide polymorphism] cluster ID [identification]), rs4646316. A Finnish group investigated the relationship of monoamines to depression in a birth cohort of 5225 patients (Nyman et al., 2011). This specific COMT mutation was associated with depression based on Hopkins Symptom Checklist-25 (HSCL) score (p=0.026). Other COMT mutations have been associated with poor responses to antidepressant treatment.

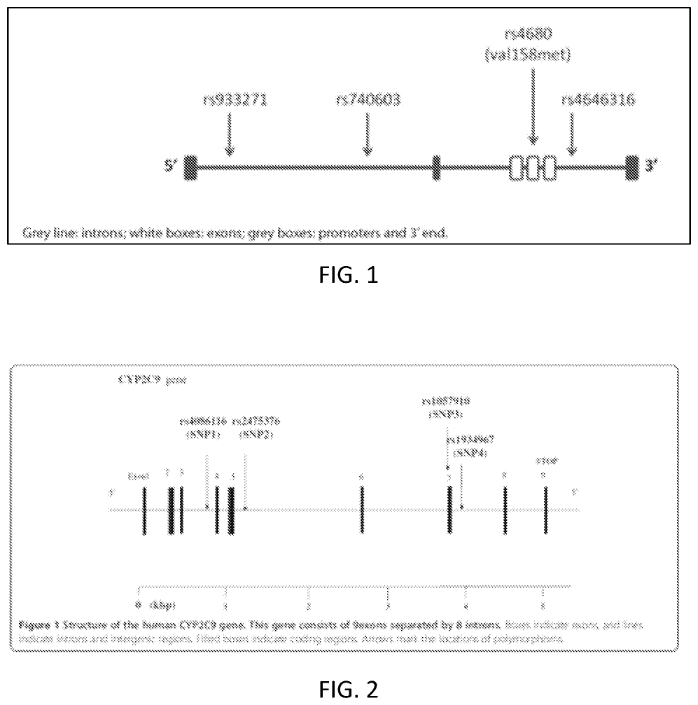

Patients in Hungary and England were investigated for COMT haplotypic variants with reference to effects on impulsivity and executive function (Pap, Gonda, et al., 2012), with statistically significant findings implicating increased impulsivity. The observed mutation is located on the intron (FIG. 1):

COMT inactivates dopamine in the prefrontal cortex (PFC), as there is a dearth of dopamine transporter in that location. Enzymatic hypofunction can be linked to deficits in working memory, executive functions, cognitive flexibility, and the ability to inhibit behavioral impulses. COMT has additionally been linked to attention-deficit hyperactivity disorder, obsessive-compulsive behavior, addiction, anxiety and psychosis. Such PFC hypofunction would explain some of the phenomenological traits observed in CHS patients.

In a related study (Pap, Juhasz, & Bagdy, 2012), COMT mutations were examined with reference to ruminative behavior, a risk factor for depression. Once again, the rs4646316 variant was examined among others in a Hungarian population, demonstrating a strong correlation to Ruminative Response Scale scores (p=0.028). The authors pointed out the role that dopamine in the PFC plays in controlling frontal cortex, as well as amygdala, striatum and hippocampus that could lead to an “impulsive cognitive style.” The hypoactive COMT mutations were hypothesized to increase dopamine in the PFC and promote rumination, which promotes rigidity and inflexibility that parallel the observations of fixed behaviors in CHS patients: prolonged employment of high-THC cannabis despite medical warnings against its continued usage, compulsive hot-water bathing, etc.

An additional study examined 193 in-patient alcoholics for mood disturbances and tendency towards relapse (Stadlin, Ho, Daglish, & Dodd, 2014). COMT rs4646316 was associated with onset of heavy alcohol intake at a younger age in female patients.

COMT is said to moderate THC effects on memory and attention (Hryhorowicz et al., 2018), and a genotype with CH in position c.472 increased likelihood of cognitive impairment with cannabis (Henquet et al., 2006). The Val158Met mutation in COMT has been associated with psychotic symptoms and development of schizophrenia in cannabis users (Caspi et al., 2005; Henquet et al., 2009).

Haloperidol, a dopamine antagonist (mostly D2), has proven more effective as an antiemetic in treatment of CHS as compared to serotonin type-3 agents (Ruberto et al., 2020), but is far from a miracle drug, and noticeably inferior to topical capsaicin treatment. Given evidence above of excessive dopaminergic activity in CHS, its superiority to first line antiemetics is sensible in context.

Treatments for patients with a variant of COMT as provided. A treatment can include stopping cannabis consumption, administration of medication, and/or a traditional remedy. For example, treatment can include administration of cannabidiol in the absence of THC using a dosing protocol sufficient to counter the effects of the disease.

### TRPV1

TRPV1 is a receptor responding to heat, ethanol and low pH that is closely associated with pain responses. Capsaicin, which is readily absorbed through the skin to an extent greater than gastrointestinal tract, is a natural agonist and desensitizer of TRPV1, as is cannabidiol (CBD). While endocannabinoids anandamide and 2-arachidonylglycerol are also ligands, THC is not. Among other functions, TRPV1 receptor has been linked to anxiety and pain responses in the brain and mediates long-term synaptic depression (LRD) in the hippocampus. TRPV1 also controls glutamate release in the solitary tract nucleus of the brainstem affecting gut motility and secretion (Peters, McDougall, Fawley, Smith, & Andresen, 2010).

No previous studies have associated TRPV1 polymorphism with cannabis dependency (Hryhorowicz et al., 2018).

Whereas this specific rs879207 mutation was not found in National Library of Medicine-listed publications, its identification in the CHS cohort is noteworthy for both its observed roles in anxiety, pain and gut motility disturbances, and the fact that hot water bathing and clinical response to cutaneous capsaicin application are critical factors of CHS phenomenology. While the mechanism is unclear, topical capsaicin application may lead to systemic absorption that in turn reaches the GI tract and brain, ameliorating propulsion, nausea, anxiety and pain engendered by this mutation.

Treatments for patients with a variant of TRPV1 is provided. A treatment can include stopping cannabis consumption, administration of medication, and/or a traditional remedy. For example, treatment can include administration of capsaicin, CBD or other natural or synthetic agents that produce TRPV1 stimulation/desensitization using a dosing protocol sufficient to counter the effects of the disease.

### CYP2C9:

Cytochrome P450 isozyme 2C9 is a catalyst for catabolism of various drugs, and endogenous vitamin D, steroids and fatty acids, especially arachidonic acid (Fu et al., 2014), the latter being a precursor to formation of the endocannabinoids, anandamide and 2-arachidonylglycerol. Although primarily located in the liver, CYP2C9 also is found in the vasculature. Some P450 enzymes are also expressed in the brain, sometimes in greater concentrations than the liver, and can be important in responses to pharmaceuticals and expressed adverse event profiles (Miksys & Tyndale, 2002), particularly toxicities associated with neurological disorders and behavioral abnormalities (McMillan & Tyndale, 2018).

CYP2C9 is the main catabolic enzyme for THC breakdown in the liver, as well as that of its psychoactive metabolite, 11-OH-THC. Concentrations of the latter were increased in carriers of CYP2C9*3 alleles and calculated intrinsic clearances 33% compared to CYP2C9*1 carriers (Sachse-Seeboth et al., 2009). The authors of the latter study recommended that slow metabolizers would experience prolonged exposure to psychoactive effects and might consider genomic testing prior to THC exposure.

Thus, CYP2C9 function may play a role in accumulation of THC in the brain, resulting in toxicity ascribable to the biphasic dose-response tendencies of cannabinoids, i.e., a reversal of effect at elevated doses. Thus, the normally anti-emetic THC could become pro-emetic at higher dose levels. Similarly, if catabolism of 11-hydroxy-THC becomes impaired due to hypoactivity of CYP2C9, it also could exert toxic effects.

A remaining possibility is that over-exposure to THC produces a down-regulation of the CB1 receptor, causing it to turn from a partial agonist to an antagonist (Sim-Selley, 2003), a phenomenon that could be hastened by impaired metabolism.

The rs1934967 mutation was identified as homozygous in our study cohort, increasing the likelihood that it has relevance to the pathophysiology of the syndrome. It is located on the intron (FIG. 2).

The haplotype CCAC of this mutation has been linked to coronary artery disease risk in Han women in Xinjiang (p=0.016) (Fu et al., 2014). Interestingly, this mutation was homozygous in the CHS population.

Treatments for patients with a variant of CYP2C9 are provided. A treatment can include stopping cannabis consumption, administration of medication, and/or a traditional remedy. For example, treatment can include administration of CBD in the absence of THC using a dosing protocol sufficient to counter the effects of the disease.

### DRD2

DRD2 gene codes for the type-2 dopamine receptor, the target of most antipsychotic drugs via its antagonism. It has a primary role in fear memories in the pre-limbic areas (Madsen, Guerin, & Kim, 2017), and has been associated with depression and anxiety (Nyman et al., 2011). Among the strongest statistical association of genomic findings in a Finnish cohort were related to the rs4648318 intron mutation: HSCL (p=0.00005) regardless of early environmental factors, HSCL depression sub-score (p=0.0015), and HSCL anxiety sub-score (p=0.02312) (Nyman et al., 2011). Other DRD2 mutations have been associated with nicotine dependence, Tourette syndrome, tanning addiction, and persistent pain.

In this context, the combination of dopamine-2 receptor and dopamine metabolism mutations seems to point to this area as important to CHS pathophysiology and phenomenology.

Treatments for patients with a variant of DRD2 are provided. A treatment can include stopping cannabis consumption, administration of medication, and/or a traditional remedy. For example, treatment can include administration of CBD in the absence of THC, as an antipsychotic remedy proven in two Phase II randomized controlled trials, using a dosing protocol sufficient to counter the effects of the disease.

### ABCA1

ABCA1 is the gene encoding the ATP-binding cassette transporter, previously known as the cholesterol efflux regulatory protein, that affects cholesterol and phospholipid homeostasis, the latter being key to Alzheimer disease (AD) and problems associated with removal of apoE and accumulation of AP deposition (Feher et al., 2018). In a study of 431 Hungarian AD patients vs. 302 elderly cognitively normal controls, a rs2230806 mutation was over-represented in demented patients, which reached statistical significance in a “recessive model” (p=0.048). This may have important implications in this context, wherein homozygosity of the mutation was observed and could imply increased risk of development of dementia.

Additional correlations for mutations of this gene include associations with coronary artery disease, and Type-2 diabetes mellitus.

Polymorphisms in a different gene, ABCB1, have been demonstrated to alter drug pharmacokinetics, and increased cannabis dependency was noted in the 3435C allele over controls (Benyamina et al., 2009).

Treatments for patients with a variant of ABCA1 are provided. A treatment can include stopping cannabis consumption, administration of medication, and/or a traditional remedy. For example, treatment can include administration of CBD or beta-caryophyllene in the absence of THC using a dosing protocol sufficient to counter the effects of the disease.

### CRY1

CRY1, Cryptochrome 1 (Photolyase-Like), a gene involved in the circadian rhythm regulation. (Drago, Monti, De Ronchi, & Serretti, 2015) encodes a flavin adenine dinucleotide-binding protein that is a key component of the circadian core oscillator complex, which regulates the circadian clock. The encoded protein is widely conserved across plants and animals. Polymorphisms in this gene have been associated with altered sleep patterns.

There is evidence that some genetic variations harbored by CRY1 are associated with mood disorders. Clinical data have demonstrated that there are abnormalities in the circadian rhythms in patients with mood disorders and those with alcohol use disorders with CRY1 rs2287161 associated with depressive disorder (Partonen, 2012).

Soria et al. analyzed 209 SNPs of 19 genes in 335 cases with depressive disorder from a two-hospital-based sample and 440 controls from a population-based sample and found that CRY1 rs2287161 C-allele was associated with depressive disorder (Soria et al., 2010).

Circadian rhythm disruption is a component of psychotic disorders (Monti et al., 2013). Links between clock gene polymorphisms and schizophrenia have been established (Mansour et al., 2009; Zhang et al., 2011). Furthermore, the CRY1 gene is located near the possible schizophrenia-susceptibility locus (Peng, Chen, & Wei, 2007). Clock genes may be associated with dopaminergic transmission, the main pharmacological target of antipsychotic drugs (Stahl, 2013).

The antipsychotic drug haloperidol, was proven to influence the biological clock of rats, shifting the period of the rhythm (MacDonald & Meck, 2005). Mokros et al. found that haloperidol may affect expression of CRY1 and especially in low concentration, blocks D2 dopamine receptor (Mokros et al., 2016).

Hickey reports of a case of CHS that improved significantly after treatment with haloperidol in the emergency department (Hickey, Witsil, & Mycyk, 2013). Jones and Abernathy (Jones & Abernathy, 2016) reported a case of severe, refractory CHS with complete resolution of nausea and vomiting after treatment with haloperidol in the outpatient setting and Witsil reported 4 cases of CHS that failed standard emergency department therapy but improved significantly after treatment with haloperidol (Witsil & Mycyk, 2017).

Treatments for patients with a variant of CRY1 are provided. A treatment can include stopping cannabis consumption, administration of medication such as haloperidol, and/or a traditional remedy. For example, treatment can include administration of CBD without THC using a dosing protocol sufficient to counter the effects of the disease.

### EXAMPLES

The following examples are offered to illustrate, but not limit the claimed invention.

### Example 1

Experiments were done to determine genes/mutations/variants associated with CHS.

Methods: After Western IRB approval, a screening questionnaire was posted online. Kits were sent to assess the DNA of patients fulfilling CHS criteria to assess single nucleotide polymorphisms (SNPs) or other mutations as compared to controls without this disorder. Controls were selected based on the following criteria: 1) Answered “NO” to ever receiving a CHS diagnosis; 2) Have used Cannabis; and 3) Answered no to all three of having frequent vomiting, nausea, and abdominal pain.

Results: 585 people took the survey. Most were high frequency users of cannabis flower or concentrates (93%), using multiple grams/d of THC-predominant material. 15.6% carried diagnoses of cannabis dependency or addiction, and 56.6% experienced withdrawal symptoms. 87.7% of patients with diagnosis or symptoms indicative of CHS were improved after cannabis cessation, most suffering recurrence rapidly after resumption of use. 103 patients who carried formal CHS diagnosis and had consistent symptom profiles were invited to submit oral swabs for genomic testing, 40 patients returned kits for genomic analysis, 28 CHS patients and 12 controls. Findings included mutations in genes coding COMT (p=0.0009), TRPV1 (p=0.021), CYP2C9 (p=0.0414), DRD2 (p=0.027) and ABCA1 (p=0.008), providing several lines of evidence relating to CHS pathophysiology and clinical manifestations (Table 2).

TABLE 2
Table of Cannabinoid Hyperemesis Genomic Testing Results

| | | | | | | | | | CHS |
|---|---|---|---|---|---|---|---|---|---|
| | | | | | | | | Control | Patients |
| | | | | | Diplo- | Haplo- | | Having | Having |
| Gene | RSID | Mutation | Allele | Zygosity | type | type | P-value* | Variant | Variant |
| COMT | rs4646316 | Intron | C > T | Het | CGGC/ | CGGC | 0.012 | 10% | 57.1% |
| | | | | | TGGC | | | | |
| ABCA1 | rs2230806 | Synon | C > T | Hom | CTTG/ | CTTG | 0.012 | 20% | 67.9% |
| | | | | | CTTG | | | | |
| TRPV1 | rs879207 | Dwnstrm | A > G | Het | ATGG/ | ATGG | 0.015 | 30% | 71.5% |
| | | | | | GTGG | | | | |
| DRD2 | rs4648318 | Intron | T > C | Het | TCCC/ | TCCC | 0.031 | 20% | 60.7% |
| | | | | | CCCC | | | | |
| CYP2C9 | rs1934967 | Intron | C > T | Hom | CTTG/ | CTTG | 0.043 | 10% | 46.4% |
| | | | | | CTTG | | (*0.011) | | (*60%) |
| TRPV1 | rs11655540 | Intron | T > G | Het | TCAA/ | TCAA | 0.066 | 30% | 64.3% |
| | | | | | GCAA | | | | |
| COMT | rs165656 | Intron | C > T | Het | CCGG/ | CCGG | 0.069 | 20% | 53.6% |
| | | | | | TCGG | | | | |
| CYP2C19 | rs4494250 | Intron | G > A | Het | GCTT/ | GCTT | 0.069 | 20% | 53.6% |
| | | | | | ACTT | | (*0.007) | | (*75%) |
| CRY1 | rs2287161 | Dwnstrm | G > C | Het | GTCG/ | GTCG | 0.091 | 50% | 78.6% |
| | | | | | CTCG | | | | |

*P-values were obtained through a fisher exact test.
*Genes CYP2C9 and CYP2C19 have a second set of values showing when patients on PPI medication were excluded from the data This was due to suspected interactions of CYP2C9 and CYP2C19 and PPI medication.

### Example 2

A variant form of COMT is found to correlate with propensity to CHS. Candidate treatments are selected accordingly and include naturally occurring cannabinoids. Volunteer CHS patients are given doses of such cannabinoids within concentration ranges already used among Cannabis users in order to stay within ranges conventionally recognized as safe. Patient responses are noted and used to guide further research and development of effective treatments for CHS.

### Example 3

A variant form of CYP2C9 is found to correlate with propensity to CHS. Candidate treatments are selected accordingly and include naturally occurring cannabinoids. Volunteer CHS patients are given doses of such cannabinoids within concentration ranges already used among Cannabis users in order to stay within ranges conventionally recognized as safe. Patient responses are noted and used to guide further research and development of effective treatments for CHS.

### Example 4

A variant form of TRPV1 is found to correlate with propensity to CHS. In vitro studies are conducted on the variant TRPV1 receptor to identify ligands capable of interacting with the receptor to disrupt the receptor activity associated with CHS. Candidate treatments are selected accordingly and include naturally occurring cannabinoids. Volunteer CHS patients are given doses of such cannabinoids within concentration ranges already used among Cannabis users in order to stay within ranges conventionally recognized as safe. Patient responses are noted and used to guide further research and development of effective treatments for CHS.

### Example 5

A variant form of DRD2 is found to correlate with propensity to CHS. Candidate treatments are selected accordingly and include ligands that can act as TRPV1 agonists and desensitizers and/or cannabinoids. Volunteer CHS patients are given doses of such ligands and/or cannabinoids within concentration ranges already used among Cannabis users in order to stay within ranges conventionally recognized as safe. Patient responses are noted and used to guide further research and development of effective treatments for CHS.

### Example 6

A variant form of ABCA1 is found to correlate with propensity to CHS. Candidate treatments are selected accordingly and include ligands that can act as TRPV1 agonists and desensitizers and/or cannabinoids. Volunteer CHS patients are given doses of such ligands and/or cannabinoids within concentration ranges already used among Cannabis users in order to stay within ranges conventionally recognized as safe. Patient responses are noted and used to guide further research and development of effective treatments for CHS.

### Example 7

Candidate treatments are selected according to the findings of Example 1 and include ligands that can act as agonists and desensitizers and/or cannabinoids. Volunteer CHS patients are given doses of such ligands and/or cannabinoids within concentration ranges already used among Cannabis users in order to stay within ranges conventionally recognized as safe. Patient responses are noted and used to guide further research and development of effective treatments for CHS.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Each of the following references is fully incorporated by reference:

Benyamina, A., Bonhomme-Faivre, L., Picard, V., Sabbagh, A., Richard, D., Blecha, L., . . . Reynaud, M. (2009). Association between ABCB1 C3435T polymorphism and increased risk of cannabis dependence. Prog Neuropsychopharmacol Biol Psychiatry, 33(7), 1270-1274. doi:10.1016/j.pnpbp.2009.07.016

Caspi, A., Moffitt, T. E., Cannon, M., McClay, J., Murray, R., Harrington, H., . . . Craig, I. W. (2005). Moderation of the effect of adolescent-onset cannabis use on adult psychosis by a functional polymorphism in the catechol-O-methyltransferase gene: longitudinal evidence of a gene X environment interaction. Biological Psychiatry, 57, 1117-1127.

Feher, A., Giricz, Z., Juhasz, A., Pakaski, M., Janka, Z., & Kalman, J. (2018). ABCA1 rs2230805 and rs2230806 common gene variants are associated with Alzheimer's disease. Neurosci Lett, 664, 79-83. doi:10.1016/j.neulet.2017.11.027

Fu, Z., Zhu, Q., Ma, Y., Huang, D., Pan, S., Xie, X., . . . Cha, E. (2014). Diplotypes of CYP2C9 gene is associated with coronary artery disease in the Xinjiang Han population for women in China. Lipids Health Dis, 13, 143. doi:10.1186/1476-511X-13-143

Henquet, C., Rosa, A., Delespaul, P., Papiol, S., Fananas, L., van Os, J., & Myin-Germeys, I. (2009). COMT ValMet moderation of cannabis-induced psychosis: a momentary assessment study of ‘switching on’ hallucinations in the flow of daily life. Acta Psychiatr Scand, 119(2), 156-160. doi:10.1111/j.1600-0447.2008.01265.x

Henquet, C., Rosa, A., Krabbendam, L., Papiol, S., Fananas, L., Drukker, M., . . . van Os, J. (2006). An experimental study of catechol-o-methyltransferase Val158Met moderation of delta-9-tetrahydrocannabinol-induced effects on psychosis and cognition. Neuropsychopharmacology, 31(12), 2748-2757. doi:10.1038/sj.npp.1301197

Hryhorowicz, S., Walczak, M., Zakerska-Banaszak, O., Slomski, R., & Skrzypczak-Zielinska, M. (2018). Pharmacogenetics of Cannabinoids. Eur J Drug Metab Pharmacokinet, 43(1), 1-12. doi:10.1007/s13318-017-0416-z

Madsen, H. B., Guerin, A. A., & Kim, J. H. (2017). Investigating the role of dopamine receptor- and parvalbumin-expressing cells in extinction of conditioned fear. Neurobiol Learn Mem, 145, 7-17. doi:10.1016/j.nlm.2017.08.009

McMillan, D. M., & Tyndale, R. F. (2018). CYP-mediated drug metabolism in the brain impacts drug response. Pharmacol Ther, 184, 189-200. doi:10.1016/j.pharmthera.2017.10.008

Miksys, S. L., & Tyndale, R. F. (2002). Drug-metabolizing cytochrome P450s in the brain. J Psychiatry Neurosci, 27(6), 406-415. Retrieved from https://www.ncbi.nlm.nih.giv/pubmed/12491573

Nyman, E. S., Sulkava, S., Soronen, P., Miettunen, J., Loukola, A., Leppa, V., . . . Paunio, T. (2011). Interaction of early environment, gender and genes of monoamine neurotransmission in the aetiology of depression in a large population-based Finnish birth cohort. BMJ Open, 1(1), e000087. doi:10.1136/bmjopen-2011-000087

Pap, D., Gonda, X., Molnar, E., Lazary, J., Benko, A., Downey, D., . . . Juhasz, G. (2012). Genetic variants in the catechol-o-methyltransferase gene are associated with impulsivity and executive function: relevance for major depression. Am J Med Genet B Neuropsychiatr Genet, 159B(8), 928-940. doi:10.1002/ajmg.b.32098

Pap, D., Juhasz, G., & Bagdy, G. (2012). Association between the COMT gene and rumination in a Hungarian sample. Neuropsychopharmacol Hung, 14(4), 285-292. Retrieved from https://www.ncbi.nlm.nih.gov/pubmed/23269216

Peters, J. H., McDougall, S. J., Fawley, J. A., Smith, S. M., & Andresen, M. C. (2010). Primary afferent activation of thermosensitive TRPV1 triggers asynchronous glutamate release at central neurons. Neuron, 65(5), 657-669. doi:10.1016/j.neuron.2010.02.017

Ruberto, A. J., Sivilotti, M. L. A., Forrester, S., Hall, A. K., Crawford, F. M., & Day, A. G. (2020). Intravenous Haloperidol Versus Ondansetron for Cannabis Hyperemesis Syndrome (HaVOC): A Randomized, Controlled Trial. Ann Emerg Med. doi:10.1016/j.annemergmed.2020.08.021

Sachse-Seeboth, C., Pfeil, J., Sehrt, D., Meineke, I., Tzvetkov, M., Bruns, E., . . . Brockmoller, J. (2009). Interindividual variation in the pharmacokinetics of Delta9-tetrahydrocannabinol as related to genetic polymorphisms in CYP2C9. Clin Pharmacol Ther, 85(3), 273-276. doi:10.1038/clpt.2008.213

Sim-Selley, L. J. (2003). Regulation of cannabinoid CB1 receptors in the central nervous system by chronic cannabinoids. Crit Rev Neurobiol, 15(2), 91-119. Retrieved from https://www.ncbi.nlm.nih.gov/entrez/query.fcgi?cmd=Retrieve&db=PubMed&dopt=Citation&list_nids=14977366

Sorensen, C. J., DeSanto, K., Borgelt, L., Phillips, K. T., & Monte, A. A. (2017). Cannabinoid Hyperemesis Syndrome: Diagnosis, Pathophysiology, and Treatment-a Systematic Review. J Med Toxicol, 13(1), 71-87. doi:10.1007/s13181-016-0595-z

Stadlin, A., Ho, A. M., Daglish, M. R., & Dodd, P. R. (2014). Personality traits, craving and genetic associations in alcoholic in-patients. Alcohol and Alcoholism 49(S1),il.

Drago, A., Monti, B., De Ronchi, D., & Serretti, A. (2015). CRY1 Variations Impacts on the Depressive Relapse Rate in a Sample of Bipolar Patients. Psychiatry Investig, 12(1), 118-124. doi:10.4306/pi.2015.12.1.118

Hickey, J. L., Witsil, J. C., & Mycyk, M. B. (2013). Haloperidol for treatment of cannabinoid hyperemesis syndrome. Am J Emerg Med, 31(6), 1003 e1005-1006. doi:10.1016/j.ajem.2013.02.021

Jones, J. L., & Abernathy, K. E. (2016). Successful Treatment of Suspected Cannabinoid Hyperemesis Syndrome Using Haloperidol in the Outpatient Setting. Case Rep Psychiatry, 2016, 3614053. doi:10.1155/2016/3614053

MacDonald, C. J., & Meck, W. H. (2005). Differential effects of clozapine and haloperidol on interval timing in the supraseconds range. Psychopharmacology (Berl), 182(2), 232-244. doi:10.1007/s00213-005-0074-8

Mansour, H. A., Talkowski, M. E., Wood, J., Chowdari, K. V., McClain, L., Prasad, K., . . . Nimgaonkar, V. L. (2009). Association study of 21 circadian genes with bipolar I disorder, schizoaffective disorder, and schizophrenia. Bipolar Disord, 11(7), 701-710. doi:10.1111/j.1399-5618.2009.00756.x

Mokros, L., Karbownik, M. S., Nowakowska-Domagala, K., Szemraj, J., Wieteska, L., Wozniak, K., . . . Pietras, T. (2016). Haloperidol, but not olanzapine, may affect expression of PER1 and CRY1 genes in human glioblastoma cell line. Biol Rhythm Res, 47(6), 865-871. doi:10.1080/09291016.2016.1202379

Monti, J. M., BaHammam, A. S., Pandi-Perumal, S. R., Bromundt, V., Spence, D. W., Cardinali, D. P., & Brown, G. M. (2013). Sleep and circadian rhythm dysregulation in schizophrenia. Prog Neuropsychopharmacol Biol Psychiatry, 43, 209-216. doi:10.1016/j.pnpbp.2012.12.021

Partonen, T. (2012). Clock gene variants in mood and anxiety disorders. J Neural Transm (Vienna), 119(10), 1133-1145. doi:10.1007/s00702-012-0810-2

Peng, Z. W., Chen, X. G., & Wei, Z. (2007). Cryptochromel maybe a candidate gene of schizophrenia. Med Hypotheses, 69(4), 849-851. doi:10.1016/j.mehy.2007.02.003

Soria, V., Martinez-Amoros, E., Escaramis, G., Valero, J., Perez-Egea, R., Garcia, C., . . . Urretavizcaya, M. (2010). Differential association of circadian genes with mood disorders: CRY1 and NPAS2 are associated with unipolar major depression and CLOCK and VIP with bipolar disorder. Neuropsychopharmacology, 35(6), 1279-1289. doi:10.1038/npp.2009.230

Stahl, S. M. (2013). Stahl's essential psychopharmacology: neuroscientific basis and practical application (Fourth edition. ed.). Cambridge: Cambridge University Press.

Witsil, J. C., & Mycyk, M. B. (2017). Haloperidol, a Novel Treatment for Cannabinoid Hyperemesis Syndrome. Am J Ther, 24(1), e64-e67. doi:10.1097/MJT.0000000000000157

Zhang, J., Liao, G., Liu, C., Sun, L., Liu, Y., Wang, Y., . . . Wang, Z. (2011). The association of CLOCK gene T3111C polymorphism and hPER3 gene 54-nucleotide repeat polymorphism with Chinese Han people schizophrenics. Mol Biol Rep, 38(1), 349-354. doi:10.1007/s11033-010-0114-2

What is claimed is:

1. A method for diagnosing Cannabinoid hyperemesis syndrome (CHS) in a patient comprising:
obtaining a sample from a patient;
analyzing the sample for a variant sequence, marker or allele of one or more genes selected from COMT, TRPV1, CYP2C9, CYP2C19, DRD2, CRY1 and ABCA1, wherein the presence of the variant sequence, marker or allele indicates a likelihood of CHS; and
diagnosing CHS based upon presence of the variant sequence, marker, or allele.

2. A kit adapted for using the method of claim 1, comprising one or more reagents capable of identifying the variant sequence, marker, or allele.

3. A method for treating CHS in a patient comprising:
obtaining a sample from the patient;
analyzing the sample for a sequence, marker or allele of COMT, TRPV1, CYP2C9, CYP2C19, DRD2, or ABCA1 associated with CHS;
determining treatment based on the presence of the sequence, marker or allele of COMT, TRPV1, CYP2C9, CYP2C19, DRD2, or ABCA1 associated with CHS.

4. The method of claim 3, wherein the treatment comprises a competitive ligand of the receptor capable of ameliorating the symptoms.

5. (canceled)

6. (canceled)

7. The method of claim 1, wherein the sequence, marker or allele comprises:
a. a haplotype of CGGC of COMT; CTTG of ABCA1; ATGG of TRPV1; TCCC of DRD2; CTTG of CYP2C9; TCAA of TRPV1; CCGG of COMT; GCTT of CYP2C19; and/or CTCG of CRY1; and/or;
b. a diplotype of CGGC/TGGC of COMT; CTTG/CTTG of ABCA1; ATGG/GTGG of TRPV1; TCCC/CCCC of DRD2; CTTG/CTTG of CYP2C9; TCAA/GCAA of TRPV1; CCGG/TCGG of COMT; GCTT/ACTT of CYP2C19; and/or GTCG/CTCG of CRY1; and/or;
c. or a marker in useful proximity thereto.

8. The kit of claim 2, wherein the sequence, marker or allele comprises:
a. a haplotype of CGGC of COMT; CTTG of ABCA1; ATGG of TRPV1; TCCC of DRD2; CTTG of CYP2C9; TCAA of TRPV1; CCGG of COMT; GCTT of CYP2C19; and/or CTCG of CRY1; and/or;
b. CGGC/TGGC of COMT; CTTG/CTTG of ABCA1; ATGG/GTGG of TRPV1; TCCC/CCCC of DRD2; CTTG/CTTG of CYP2C9; TCAA/GCAA of TRPV1; CCGG/TCGG of COMT; GCTT/ACTT of CYP2C19; and/or GTCG/CTCG of CRY1; and/or;
c. or a marker in useful proximity thereto.

9. The method of claim 3, wherein the sequence, marker or allele comprises:
a. a haplotype of CGGC of COMT; CTTG of ABCA1; ATGG of TRPV1; TCCC of DRD2; CTTG of CYP2C9; TCAA of TRPV1; CCGG of COMT; GCTT of CYP2C19; and/or CTCG of CRY1; and/or;
b. a diplotype of CGGC/TGGC of COMT; CTTG/CTTG of ABCA1; ATGG/GTGG of TRPV1; TCCC/CCCC of DRD2; CTTG/CTTG of CYP2C9; TCAA/GCAA of TRPV1; CCGG/TCGG of COMT; GCTT/ACTT of CYP2C19; and/or GTCG/CTCG of CRY1; and/or;
c. or a marker in useful proximity thereto.
